(19) United States
(12) Patent Application Publication
(10) Pub. No.: US 2024/0197806 A1
(43) Pub. Date: Jun. 20, 2024

(54) EBELIN LACTONE OR BACOGENIN A1 ENRICHED COMPOSITION AND METHOD OF PREPARATION THEREOF

(71) Applicants: SUPPLEMENT HOLDINGS, LLC, Tempe, AZ (US); NETWORK NUTRITION PTY LIMITED, Pymble (AU)

(72) Inventors: Paul Eftang, Tempe, AZ (US); Emiel Bakker, Tempe, AZ (US); James Jursich, Tempe, AZ (US); Ryan Gorman, Kiama (AU)

(21) Appl. No.: 17/988,617

(22) Filed: Nov. 16, 2022

(51) Int. Cl. A61K 36/80

(52) U.S. Cl. CPC A61K 36/80; A61K 2236/00

(57) ABSTRACT

The present disclosure provides a method of preparing an ebelin lactone or bacogenin A1 enriched composition from acid-hydrolyzed extract of Bacopa monnieri. Also provided is a composition prepared by the method. Advantageously, the present method may yield a cost effective product containing at least 10% ebelin lactone, which may result in an entirely new subjective experience with fully hydrolyzed Bacopa monnieri extract.

### BACKGROUND

Bacopa monnieri extracts prepared by conventional methods have considerable variability. This variability leads to dose-to-dose and person-to-person differences in the effects of such extract. Because of this, achieving consistent dose-to-dose effects is difficult with the untreated extracts.

Moreover, the saponin compounds in Bacopa extracts do not appear to be directly active in the central nervous system (CNS) in the same manner as ebelin lactone and bacogenin A1, nor do they lead to the beneficial effects. Binding assays have shown that ebelin lactone binds to the 5-HT2A receptors (Ki=4.21 μM), while the other compounds in Bacopa do not. As such, while the Bacopa saponins do lead to effects after ingesting normal Bacopa extracts, these effects are very different than the ones seen with ebelin lactone and bacogenin A1. The Bacopa saponins Bacoside A3, Bacopaside X, Bacopaside II, and Bacopasaponin C do bio-convert in the human gastrointestinal tract to form jujubogenin and pseudojujubogenin, but that process is extremely variable. Similarly, both jujubogenin and pseudojujubogenin can then be bio-converted in vivo to ebelin lactone and bacogenin A1 in the gastrointestinal tract of humans consuming the extract. However, this further exacerbates the inconsistent dose-to-dose effects in Bacopa extracts.

Ramasamy et al. showed that the parent bacosides (e.g., bacoside A3, bacopaside II, bacopasaponin C, and bacopaside X) have poor intestinal absorption and are not able to cross the blood brain barrier (PLoS One. 2015; 10(5): e0126565). The first direct metabolites of the bacosides (jujubogenin and pseudojujubogenin) do, however, have good intestinal absorption and are able to cross the blood brain barrier, thus being able to produce CNS effects that are necessary for the cognitive effects of Bacopa. The next set of metabolites (formed from jujubogenin and pseudojujubogenin) are ebelin lactone and bacogenin A1. While bacogenin A1 shares the same absorption parameters as jujubogenin and pseudojujubogenin, ebelin lactone on the other hand shows very high blood brain barrier penetration, meaning it is likely the most CNS active compound related to Bacopa supplementation.

Further, some people experience lethargy when taking Bacopa. These people respond great to the memory-enhancing effects, but the daytime lethargy makes it an unusable product for them. The excessive calming effects are likely coming from the first set of metabolites, jujubogenin and pseudojujubogenin, as indicated by the relatively rapid onset of calming effects when taking Bacopa.

Synthesis of ebelin lactone and bacogenin A1 is difficult. Further, no party has previously undertaken hydrolysis of the Bacopa monnieri plant material to enrich ebelin lactone. Thus, there remains a need for effective methods of preparing ebelin lactone and/or bacogenin A1 with high purity, in particular from extracts of Bacopa monnieri.

### SUMMARY

The present disclosure provides for ebelin lactone or bacogenin A1 enriched composition and method of preparation thereof. The active ingredients produced by the present methods may have improved consistency of dose-to-dose effects and improved pharmacokinetics, which leads to stronger and more desirable effects than products prepared by conventional technologies.

In one aspect, the present disclosure provides a method for preparing an ebelin lactone or bacogenin A1 enriched composition, the method comprising contacting an acid-hydrolyzed extract of Bacopa monnieri with an antisolvent, thereby precipitating the ebelin lactone or bacogenin A1 enriched composition. In some embodiments, contacting the acid-hydrolyzed extract with the antisolvent increases pH. The antisolvent can be an aqueous antisolvent. In some embodiments, the method further comprises drying and milling the precipitate. In some embodiments, the dried and milled precipitate has an ebelin lactone content of at least 10%.

In some embodiments, the acid-hydrolyzed extract of Bacopa monnieri is prepared by:
washing pulverized Bacopa monnieri thereby forming a wash-insoluble fraction;
drying the wash-insoluble fraction to a moisture content below a predetermined value;
extracting, with a polar protic extractant, bacosides from the dried wash-insoluble fraction;
concentrating the extract, wherein the concentrated extract has a bacoside content above a predetermined value;
hydrolyzing the concentrated extract with an acidified solvent, thereby forming the acid-hydrolyzed extract of Bacopa monnieri.

In some embodiments, the acidified solvent comprises a polar protic solvent and a mineral acid. For example, the polar protic solvent can be methanol or ethanol. In some embodiments, the polar protic extractant comprises methanol. The mineral acid can be sulfuric acid. In some embodiments, the predetermined moisture content is at least 15%. In some embodiments, the predetermined bacoside content is at least 20%.

The extract can be concentrated, for example, by drying. The pulverized Bacopa monnieri can be washed with an aqueous wash. The extract can be filtered prior to concentration.

In some embodiments, the acid-hydrolyzed extract of Bacopa monnieri is prepared by;
washing, with an aqueous wash, pulverized Bacopa monnieri, thereby forming a wash-insoluble fraction;
drying the wash-insoluble fraction to a moisture content below 15%;
extracting, with a polar protic extractant comprising methanol, bacosides from the dried wash-insoluble fraction;
filtering the extract,
concentrating the filtered extract, wherein the concentrated extract has a bacoside content above 20%;
hydrolyzing the concentrated extract with an acidified solvent comprising a polar protic solvent and a mineral acid, thereby forming the acid-hydrolyzed extract of Bacopa monnieri.

In another aspect, provided is a composition prepared by a method as described herein. In some embodiments, the precipitate after drying has an ebelin lactone content of at least 10%.

In another aspect, provided is a pharmaceutical composition comprising an effective amount of a composition as described herein and a pharmaceutically acceptable carrier.

In another aspect, provided is a nutraceutical composition comprising a composition as described herein and a nutraceutically acceptable carrier.

In yet another aspect, provided is a method of treating a subject in need of ebelin lactone or bacogenin A1, the method comprising administering an effective amount of a composition as described herein to the subject.

The foregoing and other aspects and advantages of the embodiments of the present disclosure will appear from the following description. In the description, reference is made to the accompanying drawings which form a part hereof, and in which there is shown by way of illustration preferred embodiments of the disclosure. Such embodiments are illustrative only, are not intended to be limited, and do not necessarily represent the full scope of the present disclosure, however, and reference is made therefore to the claims herein for interpreting the scope of the invention. As such, features of the presently disclosed subject matter will be apparent from the following detailed description and the appended claims that follow.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows structures of bacoside A saponin glycosides and aglycones.

FIG. 2 shows formation of ebelin lactone and bacogenin A1.

FIG. 3 shows a representative HPTLC plate of ebelin lactone extract (lanes 4-8) compared to Bacopa monnieri (lanes 1-3).

FIG. 4A and FIG. 4B show representative UPLC chromatograms of ebelin lactone standardized extract at 205 nm and 278 nm, respectively.

### DETAILED DESCRIPTION OF THE INVENTION

In various embodiments, the present disclosure provides methods of preparing ebelin lactone, bacogenin A1, jujubogenin, and pseudojujubogenin with consistent quality, standardized yield, and desired specification. The active ingredients produced by the present methods may have improved consistency of dose-to-dose effects and improved pharmacokinetics, which leads to stronger and more desirable effects than products prepared by conventional technologies.

### Definitions

The present invention has been described in terms of one or more preferred embodiments, and it should be appreciated that many equivalents, alternatives, variations, and modifications, aside from those expressly stated, are possible and within the scope of the invention. Before the present invention is described, it is understood that this invention is not limited to the particular methodology, protocols, and reagents described, as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

The terms“comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modem Methods of Organic Synthesis, 3^{rd} Edition, Cambridge University Press, Cambridge, 1987; the entire contents of each of which are incorporated herein by reference.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

### Method of Preparation

The present disclosure relates to methods of preparing a product from extract of Bacopa monnieri (Brahmi), which is a traditionally used Indian herb. In conventional extracts of Bacopa monnieri, the target constituents are bacosides. Ebelin lactone would not be detectable in a conventional extract of Bacopa monnieri and would instead be prepared in vivo. In contrast, the present disclosure provides a manufacturing process for hydrolyzing constituents available in the raw herb to biologically available ebelin lactone and/or bacogenin A1. Thus, the present disclosure provides compositions having a content of ebelin lactone and/or bacogenin A1 that can be readily formulated into pharmaceutical and dietary supplement forms for consumption.

Bacopa monnieri has been used in Ayurvedic medicine to improve memory and cognition. The active constituents responsible for its pharmacological effects include bacoside A, a mixture of dammarane-type triterpenoid saponins containing sugar chains linked to a steroid aglycone skeleton. Triterpenoid saponins have been reported to be transformed in vivo to metabolites that give better biological activity and pharmacokinetic characteristics. Bacopa monnieri extract may improve cognition and has pharmacological activities such as antiepileptic, anxiolytic, antidepressant, sedative, antioxidant and anti-inflammatory activities. The cognitive effects of Bacopa monnieri extract and metabolites/derivatives thereof may be attributed to their role in acetylcholinesterase (AChE) inhibition, β-amyloid reduction, antioxidant neuroprotection, neurotransmitter modulation (acetylcholine [ACh], 5-hydroxytryptamine [5-HT], dopamine [DA]), choline acetyltransferase activation and increased cerebral blood flow.

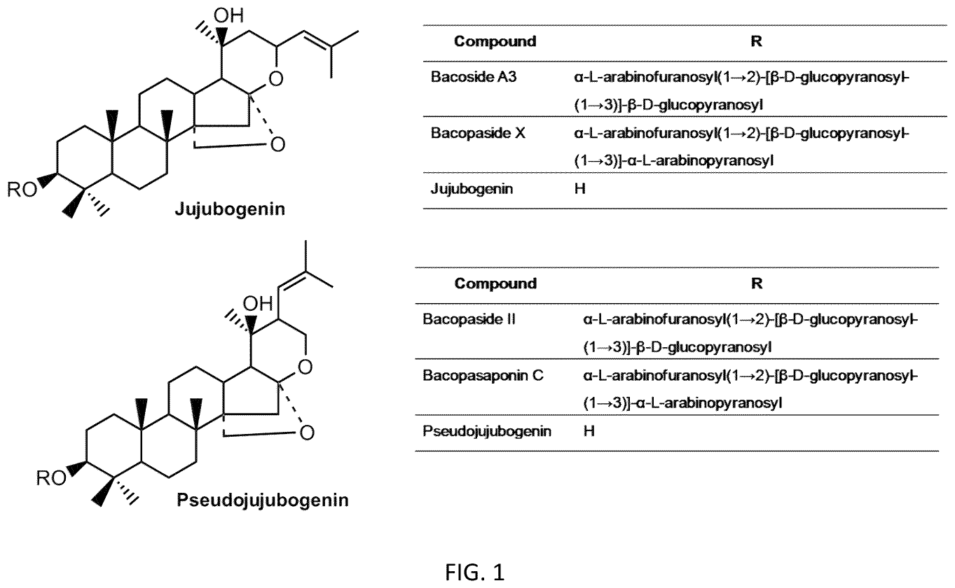

Bacoside A is a mixture of four triglycosidic saponins, namely bacoside A3, bacopaside II, bacopasaponin C and the jujubogenin isomer of bacosaponin C (bacopaside X). These bacosides are dammarane types of triterpenoid saponins with jujubogenin or pseudojujubogenin moieties as the aglycone units (FIG. 1).

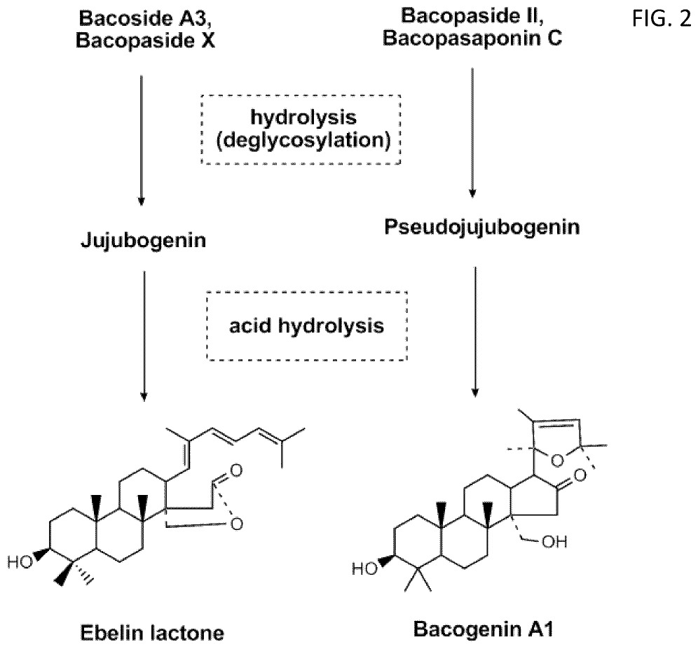

The acid hydrolysis may release the aglycone units from the parent bacosides (e.g., in a deglycosylation step). The acid hydrolysis may also convert jujubogenin and pseudojujobogenin into ebelin lactone and bacogenin A1, respectively (FIG. 2). In some embodiments, the acid-hydrolyzed extract of Bacopa monnieri comprises aglycones, such as ebelin lactone and bacogenin A1.

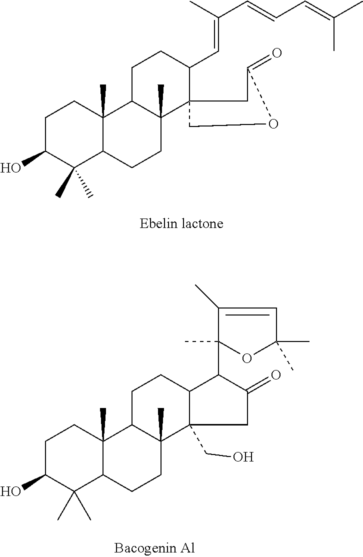

Without wishing to be bound by theory, it is believed that two mechanistic pathways are possible: a “single step” pathway whereby jujubogenin protonation leads to a ring collapse and subsequent formation of Ebelin lactone (Scheme 1), and a “multi-step” pathway whereby multiple protonation and dehydration steps occur sequentially to yield the final ebelin lactone product (Scheme 2). Both mechanisms may occur concomitantly under acidic conditions, where the relative reaction rates drive the predominance of one mechanism over another.

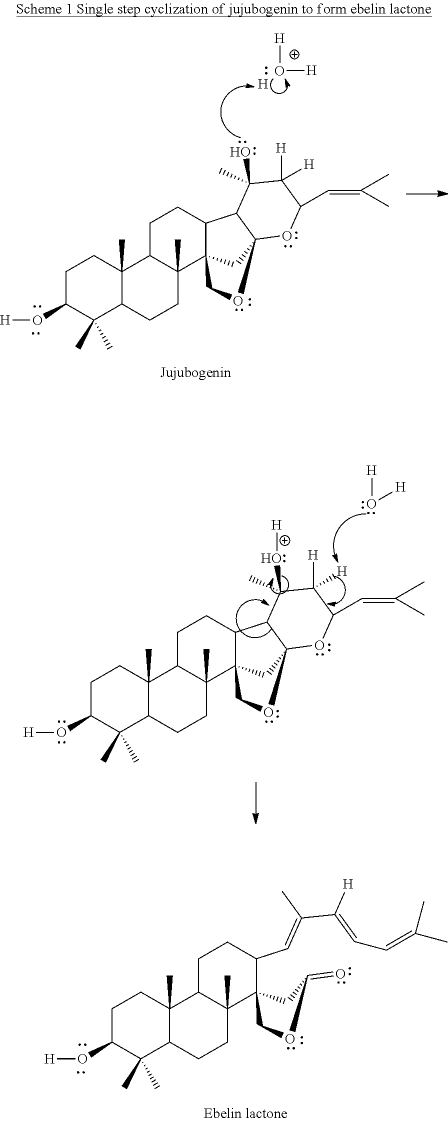

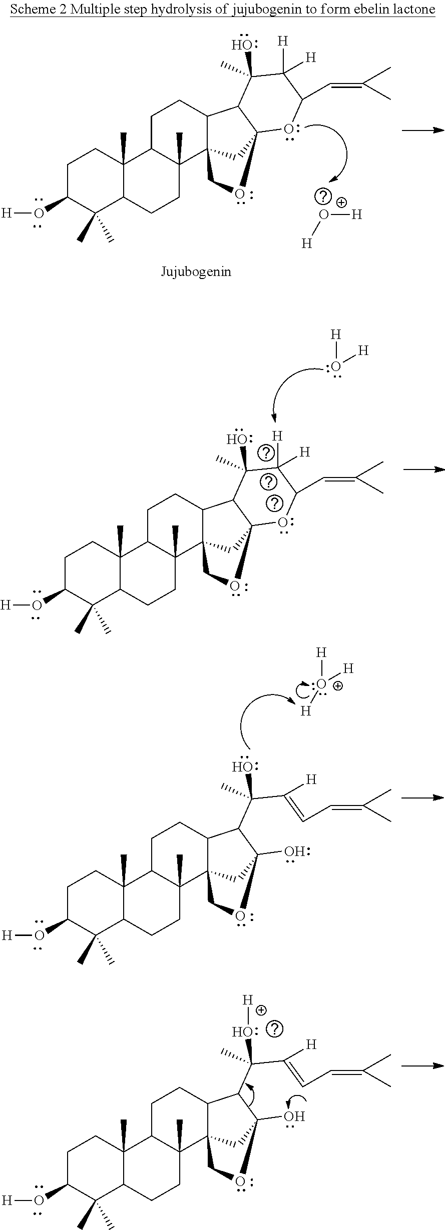

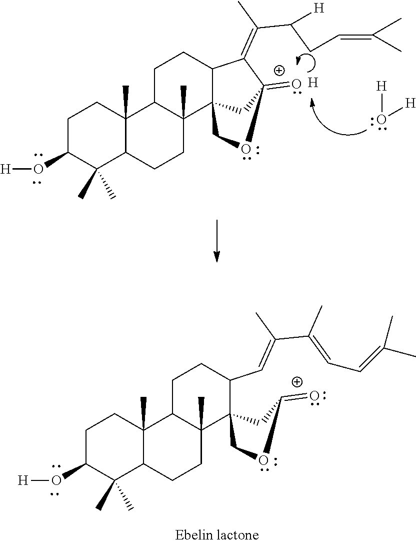

In one aspect, the present disclosure provides a method for preparing an ebelin lactone or bacogenin A1 enriched composition, the method comprising contacting an acid-hydrolyzed extract of Bacopa monnieri with an antisolvent, thereby precipitating the ebelin lactone or bacogenin A1 enriched composition.

An antisolvent is a solvent in which a solute is less soluble than a first solvent. Accordingly, addition of an effective amount of an antisolvent to a solution comprising the solute and first solvent results in the solute precipitating out of solution.

Extract of Bacopa monnieri may be prepared by extraction processes known in the art, such as the process described in WO 2017/103831, which is incorporated herein by reference in its entirety. The extraction process may include, for example, harvesting and cleaning Bacopa monnieri followed by drying, grinding, and pulverizing the cleaned herb to provide a powder.

In some embodiments, the process comprises washing pulverized Bacopa monnieri, thereby forming a wash-insoluble fraction. For example, the pulverization may be performed using a mechanical mill to reduce the crude herb to a coarse powder (e.g., NLT 40 mesh particle size). The pulverized powder may be washed, for example, by an aqueous wash (such as water or a solution in water).

In some embodiments, the process further comprises drying the wash-insoluble fraction to a moisture content below a predetermined value. For example, moisture analysis may be conducted as standard oven drying at 105° C. for 2-3 hours. Moisture may be controlled at a level of at most 15%. The moisture level of the intermediate material may be at most 14%, at most 12%, at most 10%, at most 8%, at most 6%, at most 4%, at most 2%, at most 1%, at most 0.5%, at most 0.1%, or at most 0.01%. The moisture level may be less than 15%, less than 14%, less than 12%, less than 10%, less than 8%, less than 6%, less than 4%, less than 2%, less than 1%, less than 0.5%, less than 0.1%, or less than 0.01%.

The active constituents may be extracted from Bacopa monnieri powder with a polar protic extractant. Suitably the polar protic extractant may be an alcohol (e.g., methanol or ethanol). In some embodiments, the process comprises extracting, with a polar protic extractant, bacosides from the dried wash-insoluble fraction. In some embodiments, the process uses methanol and/or ethanol as extractant. In some embodiments, the process uses methanol only as extractant.

The extraction process may be carried out, for example, at a temperature range of about 40-80° C., such as about 40° C., about 50° C., about 60° C., about 70° C., or about 80° C. The extract may then be collected, and the volume of the extract may be reduced by removing the extractant (e.g., an alcohol), for example by evaporation to provide a concentrated extract. In some embodiments, the process comprises concentrating the extract, wherein the concentrated extract has a bacoside content above a predetermined value. In some embodiments, the concentrated extract (or a pre-hydrolysis material) contains at least 20% bacosides by weight (% w/w calculated as-is) to achieve a target minimum of 10% ebelin lactone and/or 10% bacogenin A1 in the final extract. A lower bacosides content may influence the yield or quality of ebelin lactone and/or bacogenin A1 in the final extract, which may result in a lower potency. The concentrated extract (or a pre-hydrolysis material) may contain at least 25%, at least 30%, at least 35%, at least 40%, at least 45%, or at least 50% bacosides by weight.

In some embodiments, the Bacopa monnieri extract is filtered to remove insoluble components.

The Bacopa monnieri extract is subject to acid hydrolysis. In some embodiments, the herbal extract is mixed with a mineral acid. Suitable mineral acids include, but are not limited to hydrochloric acid, sulfuric acid, orthophosphoric acid, and a combination thereof. The concentration of the mineral acid may be, for example, 10% v/v, 20% v/v, or 30% v/v. In some embodiments, the mineral acid is hydrochloric acid at 20% v/v. The acid hydrolysis may be carried out at a temperature of at least 30° C. The duration of the acid hydrolysis may be at least 2 hours. In some embodiments, the mixture of mineral acid and concentrated herbal extract of Bacopa monnieri may be stirred for 5-6 hours at a temperature range of 40-70° C. to perform the acid hydrolysis.

In some embodiments, the process comprises hydrolyzing the concentrated extract with an acidified solvent, thereby forming the acid-hydrolyzed extract of Bacopa monnieri. In some embodiments, the acidified solvent comprises a polar protic solvent and a mineral acid. The polar protic solvent may be water, an alcohol, such as methanol or ethanol, or a mixture thereof. In some embodiments, the present process uses sulfuric acid (e.g., 2N) in a water/alcohol mixture as the acidified solvent. The water/alcohol mixture may be, for example, a water/methanol or water/ethanol mixture. The water:alcohol ratio in the water/alcohol mixture may be about 30%:70%, about 40%:60%, about 50%:50%, about 60%:40%, or about 70%:30%. In some embodiments, the acidified solvent is water/methanol (50%:50%) containing 2N sulfuric acid. After hydrolysis, the extract solution may be cooled to room temperature and washed with water (e.g., until pH reaches 7.0).

The acid-hydrolyzed extract may comprise an organic solvent. The organic solvent may a protic solvent or an aprotic solvent. The organic solvent may be, for example, an alcohol (e.g., methanol, ethanol, isopropanol), acetone, acetonitrile, dimethylformamide (DMF), dimethyl sulfoxide (DMSO), or a combination thereof.

The antisolvent may be an aqueous antisolvent. The aqueous solution may comprise water and optionally one or more organic solvents. The organic solvents in the antisolvent may be a poor solvent to dissolve ebelin lactone and/or bacogenin A1. In some embodiments, the antisolvent comprises water and a poor organic solvent. In some embodiments, the antisolvent is water or a solution in water. The antisolvent (e.g., a solution in water) may comprise a base for adjusting pH.

In some embodiments, contacting the acid-hydrolyzed extract with the antisolvent increases pH of the acid-hydrolyzed extract. The resulting pH after contacting the acid-hydrolyzed extract with the antisolvent may be, for example, about 6.0, about 6.2, about 6.4, about 6.6, about 6.8, about 7.0, about 7.2, about 7.4, about 7.6, about 7.8, or about 8.0. In some embodiments, the resulting pH after contacting the acid-hydrolyzed extract with the antisolvent is about 7.0.

The precipitated ebelin lactone or bacogenin A1 enriched composition may be a solid or semisolid, which can be separated from the liquid phase of solvents. In some embodiments, the method further comprises drying and milling the precipitate. The moisture content of the dried precipitate may be, for example, less than 5%, less than 2%, less than 1%, less than 0.1%, or less than 0.01% by weight. The precipitate may be in a form of particles (e.g., after milling). The size of the precipitate (e.g., a diameter of the particles) is not limited. For example, the size of the precipitate may be about 0.1 μm, about 1 μm, about 10 μm, about 100 μm, or about 1 mm.

The content of ebelin lactone and/or bacogenin A1 in the precipitated composition may be determined using any suitable analytical technique, including for example HPLC-UV/Photodiode Array Detector (PDA). Bacoside standards (bacoside A3) may be used and subjected to hydrolysis for analyzing ebelin lactone and/or bacogenin A1 contents. In some embodiments, the dried (and optionally the milled) precipitate has an ebelin lactone content of at least 10% by weight. The ebelin lactone content may be at least 15%, at least 20%, at least 25%, at least 30%, at least 35%, or at least 40% by weight. In some embodiments, the dried (and optionally the milled) precipitate has a bacogenin A1 content of at least 10% by weight. The bacogenin A1 content may be at least 15%, at least 20%, at least 25%, at least 30%, at least 35%, or at least 40% by weight.

In one embodiment, the acid-hydrolyzed extract of Bacopa monnieri is prepared by:
washing pulverized Bacopa monnieri, thereby forming a wash-insoluble fraction;
drying the wash-insoluble fraction to a moisture content below a predetermined value;
extracting, with a polar protic extractant, bacosides from the dried wash-insoluble fraction;
concentrating the extract, wherein the concentrated extract has a bacoside content above a predetermined value; and
hydrolyzing the concentrated extract with an acidified solvent, thereby forming the acid-hydrolyzed extract of Bacopa monnieri.

Each step of the preparing the acid-hydrolyzed extract above may be carried out as described herein. The acid-hydrolyzed extract of Bacopa monnieri is then subject to contacting with an antisolvent as described herein, thereby precipitating the ebelin lactone or bacogenin A1 enriched composition. Contacting the acid-hydrolyzed extract with the antisolvent may increases pH of the extract. In some embodiments, the antisolvent is an aqueous antisolvent, such as water.

In some embodiments, the acidified solvent comprises a polar protic solvent and a mineral acid. In some embodiments, the polar protic solvent is methanol or ethanol. In some embodiments, the mineral acid is sulfuric acid.

In some embodiments, the predetermined moisture content is at most 15%. In some embodiments, the polar protic extractant comprises methanol.

In some embodiments, the predetermined bacoside content is at least 20%. In some embodiments, the extract is concentrated by drying (e.g., removing the extractant by evaporation).

In some embodiments, the pulverized Bacopa monnieri is washed with an aqueous wash. In some embodiments, the extract is filtered prior to concentration.

In another embodiment, the acid-hydrolyzed extract of Bacopa monnieri is prepared by:
washing, with an aqueous wash, pulverized Bacopa monnieri, thereby forming a wash-insoluble fraction;
drying the wash-insoluble fraction to a moisture content below 15%;
extracting, with a polar protic extractant comprising methanol, bacosides from the dried wash-insoluble fraction;
filtering the extract;
concentrating the filtered extract, wherein the concentrated extract has a bacoside content above 20%; and
hydrolyzing the concentrated extract with an acidified solvent comprising a polar protic solvent and a mineral acid, thereby forming the acid-hydrolyzed extract of Bacopa monnieri.

### Composition

In another aspect, provided is an ebelin lactone or bacogenin A1 enriched composition. The composition may be obtained from Bacopa monnieri extracts, including acid-hydrolyzed extracts. In some embodiments, the composition is prepared by the method for preparing an ebelin lactone or bacogenin A1 enriched composition as described here.

In some embodiments, the precipitate after drying has an ebelin lactone content of at least 10% by weight. The ebelin lactone content may be at least 15%, at least 20%, at least 25%, at least 30%, at least 35%, or at least 40% by weight. In some embodiments, the precipitate after drying has a bacogenin A1 content of at least 10% by weight. The bacogenin A1 content may be at least 15%, at least 20%, at least 25%, at least 30%, at least 35%, or at least 40% by weight.

In one embodiment, the composition is obtained from an acid-hydrolyzed extract of Bacopa monnieri, which is prepared by:
washing pulverized Bacopa monnieri, thereby forming a wash-insoluble fraction;
drying the wash-insoluble fraction to a moisture content below a predetermined value;
extracting, with a polar protic extractant, bacosides from the dried wash-insoluble fraction;
concentrating the extract, wherein the concentrated extract has a bacoside content above a predetermined value; and
hydrolyzing the concentrated extract with an acidified solvent, thereby forming the acid-hydrolyzed extract of Bacopa monnieri.

In another embodiment, the composition is obtained from an acid-hydrolyzed extract of Bacopa monnieri, which is prepared by:
washing, with an aqueous wash, pulverized Bacopa monnieri, thereby forming a wash-insoluble fraction;
drying the wash-insoluble fraction to a moisture content below 15%;
extracting, with a polar protic extractant comprising methanol, bacosides from the dried wash-insoluble fraction;
filtering the extract;
concentrating the filtered extract, wherein the concentrated extract has a bacoside content above 20%; and
hydrolyzing the concentrated extract with an acidified solvent comprising a polar protic solvent and a mineral acid, thereby forming the acid-hydrolyzed extract of Bacopa monnieri.

The acid-hydrolyzed extract of Bacopa monnieri then may be subject to contacting with an antisolvent as described herein, thereby precipitating the ebelin lactone or bacogenin A1 enriched composition. In these embodiments, the precipitate after drying may have an ebelin lactone and/or bacogenin A1 content of at least 10%.

The amounts of active components of the Bacopa monnieri extract, such as ebelin lactone or bacogenin A1, in the present composition may be different in various embodiments. The composition may comprise ebelin lactone in an amount of at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, or at least 50% by weight. The composition may comprise bacogenin A1 in an amount of at least 0.5%, at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 20%, at least 25%, at least 30%, at least 35%, at least 40%, at least 45%, or at least 50% by weight.

Pharmaceutical and/or nutraceutical compositions may be prepared from the compositions prepared from the methods described herein.

In some embodiments, the composition is a nutraceutical composition comprising at least one extract of Bacopa monnieri as disclosed herein, and at least one nutraceutically acceptable carrier. As used herein, “nutraceutical composition” is a substance that provides a medical or health benefit, including the prevention and/or treatment of disease. Nutraceutical compositions may comprise a vitamin, a mineral, an herb or other botanical, an amino acid, a dietary substance, or a concentrate, metabolite, constituent, extract, or any combination thereof.

In some embodiments, the composition is a pharmaceutical composition comprising at least one extract of Bacopa monnieri as disclosed herein, and at least one pharmaceutically acceptable carrier.

The term “pharmaceutically acceptable” or “nutraceutically acceptable,” as used herein, refers to any carrier, excipient, or ingredient which is useful in the preparation of a pharmaceutical or nutraceutical composition, which is generally safe, non-toxic and neither biologically nor otherwise undesirable, and which is acceptable for veterinary use as well as for human application.

The pharmaceutically or nutraceutically acceptable carrier may be a non-toxic, inert solid, semi-solid or liquid filler, diluent, encapsulating material or formulation auxiliary of any type. Suitable carriers include, but are not limited to, diluents, preservatives, solubilizers, emulsifiers, liposomes, nanoparticles and adjuvants. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Compositions of the present disclosure may include liquids, lyophilized, or otherwise dried formulations and may include diluents of various buffer content (e.g., Tris-HCl, acetate, phosphate), pH and ionic strength, additives such as albumin or gelatin to prevent absorption to surfaces, detergents (e. g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the polypeptide, complexation with metal ions, or incorporation of the material into or onto particulate preparations of polymeric compounds such as polylactic acid, polyglycolic acid, hydrogels, etc., or onto liposomes, microemulsions, micelles, or milamellar or multilamellar vesicles. Such compositions will influence the physical state, solubility, stability, rate of in vivo release, and rate of in vivo clearance. Controlled or sustained release compositions include formulation in lipophilic depots (e.g., fatty acids, waxes, oils).

The compositions can be sterilized by conventional, well-known sterilization techniques. The compositions may contain pharmaceutically or nutraceutically acceptable additional substances as required to approximate physiological conditions such as a pH adjusting and buffering agent, toxicity adjusting agents, such as, sodium acetate, sodium chloride, potassium chloride, calcium chloride, sodium lactate, and the like.

The present compositions may be formulated for administration by, for example, solid dosing, eyedrop, in a topical oil-based formulation, injection, inhalation (either through the mouth or the nose), implants, oral, buccal, parenteral, or rectal administration. Techniques and formulations may generally be found in “Reminington's Pharmaceutical Sciences”, (Meade Publishing Co., Easton, Pa.). Therapeutic compositions typically are sterile and stable under the conditions of manufacture and storage.

The route by which the present composition is administered and the form of the composition may dictate the type of carrier to be used. The composition may be administered, for example, by oral, rectal, sublingual, parenteral, or topical administration. Parenteral administration may include, for example, intramuscular, intraperitoneal, intravenous, and transdermal administration. The composition may be in a variety of forms, suitable, for example, for systemic administration (e.g., oral, rectal, sublingual, buccal, implants, or parenteral) or topical administration (e.g., dermal, pulmonary, nasal, aural, ocular, liposome delivery systems, or iontophoresis).

Carriers for systemic administration may include at least one of diluents, lubricants, binders, disintegrants, colorants, flavors, sweeteners, antioxidants, preservatives, glidants, solvents, suspending agents, wetting agents, surfactants, combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. The amount of diluent(s) in a systemic or topical composition is typically about 50 to about 90%.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. The amount of lubricant(s) in a systemic or topical composition is typically about 5 to about 10%.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. The amount of binder(s) in a systemic composition is typically about 5 to about 50%.

Suitable disintegrants include agar, alginic acid and the sodium salt thereof, effervescent mixtures, croscarmelose, crospovidone, sodium carboxymethyl starch, sodium starch glycolate, clays, and ion exchange resins. The amount of disintegrant(s) in a systemic or topical composition is typically about 0.1 to about 10%.

Suitable colorants include a colorant such as an FD&C dye. When used, the amount of colorant in a systemic or topical composition is typically about 0.005 to about 0.1%.

Suitable flavors include menthol, peppermint, and fruit flavors. The amount of flavor(s), when used, in a systemic or topical composition is typically about 0.1 to about 1.0%.

Suitable sweeteners include aspartame and saccharin. The amount of sweetener(s) in a systemic or topical composition is typically about 0.001 to about 1%.

Suitable antioxidants include butylated hydroxyanisole (“BHA”), butylated hydroxytoluene (“BHT”), and vitamin E. The amount of antioxidant(s) in a systemic or topical composition is typically about 0.1 to about 5%.

Suitable preservatives include benzalkonium chloride, methyl paraben and sodium benzoate. The amount of preservative(s) in a systemic or topical composition is typically about 0.01 to about 5%.

Suitable glidants include silicon dioxide. The amount of glidant(s) in a systemic or topical composition is typically about 1 to about 5%.

Suitable solvents include water, isotonic saline, ethyl oleate, glycerine, hydroxylated castor oils, alcohols such as ethanol, and phosphate buffer solutions. The amount of solvent(s) in a systemic or topical composition is typically from about 0 to about 100%.

Suitable suspending agents include AVICEL RC-591 (from FMC Corporation of Philadelphia, Pa.) and sodium alginate. The amount of suspending agent(s) in a systemic or topical composition is typically about 1 to about 8%.

Suitable surfactants include lecithin, Polysorbate 80, and sodium lauryl sulfate, and the TWEENS from Atlas Powder Company of Wilmington, Del. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239. The amount of surfactant(s) in the systemic or topical composition is typically about 0.1% to about 5%.

Although the amounts of components in the systemic compositions may vary depending on the type of systemic composition prepared, in general, systemic compositions include 0.01% to 50% of active agent and 50% to 99.99% of one or more carriers. Compositions for parenteral administration typically include 0.1% to 10% of actives and 90% to 99.9% of a carrier including a diluent and a solvent.

Compositions for oral administration can have various dosage forms. For example, solid forms include tablets, capsules, granules, and bulk powders. These oral dosage forms include a safe and effective amount, usually at least about 5%, and more particularly from about 25% to about 50% of actives. The oral dosage compositions include about 50% to about 95% of carriers, and more particularly, from about 50% to about 75%.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include an active compound, and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

The selection of ingredients in the carrier for oral compositions depends on secondary considerations like taste, cost, and shelf stability, which are not limited for the present compositions.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT coatings (available from Rohm & Haas G.M.B.H. of Darmstadt, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

### Method of Use

The extracts and compositions as disclosed herein may be used to treat a disease or disorder. In one aspect, the disclosure provides a method of treating subject in need of ebelin lactone or bacogenin A1, the method comprising administering an effective amount of an extract or a composition as disclosed herein to the subject.

In some embodiments, the subject is in need of a neuroprotectant. Neuroprotectants serve to prevent neuron damage, degeneration, or impairment of function or alleviate symptoms or complications of neuron damage, degeneration, or impairment of function. The neuroprotective effect of the presently disclosed compounds may be associated with improved memory and/or cognition. The neuroprotectant may comprise an effective amount of an extract of composition as disclosed herein.

In some embodiments, the subject is in need of an anticonvulsant. The anticonvulsant may comprise and effective amount of an extract of composition as disclosed herein.

In some embodiments, the subject is in need of an antidepressant. The antidepressant may comprise and effective amount of an extract of composition as disclosed herein.

In some embodiments, the subject is in need of an anxiolytic. The anxiolytic may comprise and effective amount of an extract of composition as disclosed herein.

In some embodiments, the subject is in need of a sedative. The sedative may comprise and effective amount of an extract of composition as disclosed herein.

In some embodiments, the subject is need of an adaptogen. The adaptogen may comprise and effective amount of an extract of composition as disclosed herein.

In some embodiments, the subject is in need of an anti-inflammatory. The anti-inflammatory may comprise and effective amount of an extract of composition as disclosed herein.

In some embodiments, the subject is in need of an antioxidant. The antioxidant may comprise and effective amount of an extract of composition as disclosed herein.

The “effective amount” or “therapeutically effective amount” or a “sufficient amount” as used herein includes pharmaceutically or nutraceutically effective amount, which refers to a quantity effective, at dosages and for periods of time necessary, to achieve a desired result, such as beneficial or desirable biological and/or clinical results. The effective amount of the composition may be determined by a person skilled in the art and may vary according to factors such as the disease state, age, sex, and weight of the individual, and the ability of the composition to elicit a desired response in the individual. Also, the “effective amount” may be an amount resulting in a beneficial or desired result in a subject as compared to a control. The effective amount of an extract or composition of the present disclosure may be readily determined by one of ordinary skill by routine methods known in the art. Dosage regime may be adjusted to provide the optimum therapeutic response. The effective amount also may be one in which any toxic or detrimental effects of the present composition are outweighed by the therapeutically beneficial effects.

For example, an effective amount of the present composition may be about 1 mg/kg to about 1000 mg/kg, such as about 5 mg/kg to about 1000 mg/kg, about 10 mg/kg to about 1000 mg/kg, about 10 mg/kg to about 800 mg/kg, about 10 mg/kg to about 400 mg/kg, about 10 mg/kg to about 200 mg/kg, about 10 mg/kg to about 100 mg/kg, about 20 mg/kg to about 800 mg/kg, about 20 mg/kg to about 400 mg/kg, about 20 mg/kg to about 200 mg/kg, about 30 mg/kg to about 600 mg/kg, or about 30 mg/kg to about 300 mg/kg. The effective amount may be, for example, about 1 mg/kg, about 10 mg/kg, about 20 mg/kg, about 50 mg/kg, about 100 mg/kg, about 200 mg/kg, about 500 mg/kg, or about 800 mg/kg.

As used herein, “subject” or “patient” refers to mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex. In one specific embodiment, a subject is a mammal, preferably a human. In some embodiments, the subject is a human suffering from a disease or disorder needing therapeutic treatment. Suitable subjects may have a disease or disorder, specifically a disease or disorder that can be treated by an extract or composition as described herein. The subject may be a mammal, in particular a human.

The term “treating” or “treatment” as used herein describes the management and care of a subject for combating a disease, condition, or disorder. Treating includes the administration of an active agent (such as an extract or composition as disclosed herein) for preventing the onset of the symptoms or complications, alleviating the symptoms or complications, or eliminating the disease, condition, or disorder. As used herein, and as well understood in the art, “treatment” or “treating” is also an approach for obtaining beneficial or desired results, including clinical results. Beneficial or desired clinical results can include, but are not limited to, alleviation or amelioration of one or more symptoms or conditions, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment. Further, any of the treatment methods or uses described herein can be formulated alone or for contemporaneous administration with other agents or therapies.

In some embodiments, the subject is in need of a treatment for a neurodegenerative disease, such as Alzheimer's disease, Parkinson's disease, or dementia. Subjects in need of a treatment for a neurodegenerative disease may be administered a neuroprotectant as described herein.

In some embodiments, the subject is in need of a treatment for epilepsy. Subjects in need of a treatment for epilepsy may be administered an anticonvulsant as described herein.

In some embodiments, the subject is in need of a treatment for depression. Subjects in need of a treatment for depression may be administered an antidepressant as described herein.

In some embodiments, the subject is in need of a treatment for anxiety. Subjects in need of a treatment for anxiety may be administered an anxiolytic, sedative, or adaptogen as described herein.

In some embodiments, the subject is in need of a treatment for inflammation. Subjects in need of a treatment for an inflammation may be administered an anti-inflammatory as described herein.

In some embodiments, the subject is in need of a treatment for attention deficit-hyperactivity disorder (ADHD). Subjects in need of a treatment for an ADHD may be administered an effective amount of an extract of composition as disclosed herein as described herein to improve memory and/or cognition.

As used herein, the terms “administering” and “administration” refer to any method of providing a pharmaceutical preparation or composition to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, intradermal administration, intrathecal administration and subcutaneous administration. Administration can be continuous or intermittent. In various aspects, a preparation can be administered therapeutically; that is, administered to treat an existing disease or condition. In some embodiments, the administration is intravenous administration.

It should be apparent to those skilled in the art that many additional modifications beside those already described are possible without departing from the inventive concepts. Aspects of the present disclosure that are described with respect to methods can be utilized in the context of the compositions of matter discussed in this disclosure. Similarly, aspects of the present disclosure that are described with respect to compositions of matter can be utilized in the context of the methods. The invention will be more fully understood upon consideration of the following non-limiting examples.

### EXAMPLES

### Example 1: Extraction and Preparation

An extract of Bacopa monnieri (Brahmi), which is a traditionally used Indian herb, was provided employing a novel manufacturing process for hydrolyzing constituents available in the raw herb to biologically available ebelin lactone. In typical extracts of Bacopa monnieri, the target constituents are bacosides. In the preparation described herein, bacosides were hydrolyzed into their more biologically available form. Ebelin lactone would not be detectable in a typical extract of Bacopa monnieri and would instead hydrolyze in the human system. The present process delivers a content of ebelin lactone in the final extract which can be readily formulated into pharmaceutical and dietary supplement forms for convenient consumption.

Authenticated whole plant of Bacopa monnieri was selected as the material to be process. Bacopa monnieri, also known as Brahmi or commonly known as Water Hyssop, is an herb native to India and several other countries. Once the starting material is qualified as authentic Bacopa monnieri whole plant, it is advanced to pre-production stage where it is pulverized into a coarse powder ready for extraction.

The coarse powder was subjected to two stages of washing with water 80-85° C. for at least 4 hours, then filtered. The water soluble components are discarded from the process and the water insoluble fraction is retained, and dried until moisture content reduces below 15%.

The water insoluble fraction with reduced moisture is then extracted in methanol at 70-75° C. for 3 hours. Methanol extraction of the herb material is repeated two more times then passed through a 5 micron filter and concentrated at 70-75° C. to remove methanol.

The concentrated extract is then dried at 70-75° C. in a vacuum tray drier. The dried extract was tested and confirmed to contain not less than 20% bacosides when tested using the method described in the United States Pharmacopoeia monograph (Powdered Bacopa Extract) for content of triterpene glycosides.

The dried powdered extract containing no less than 20% bacosides was then hydrolyzed with acidified methanol (or ethanol)/water combination (50% methanol, 50% water, with addition of 2N sulfuric acid). The hydrolysis takes place over 4 hours of exposure to the acidified hydro-methanolic combination at 80-85° C.

After four hours of hydrolysis, the material was allowed to cool to ambient temperature, then water is added until pH reaches 7.0, forming a precipitate. The precipitate is collected and dried at 75-80° C. in a vacuum tray drier, before being milled to the required particle size and presented as a powder.

The final powder was tested to confirm presence and content of ebelin lactone at not less than 10% using a proprietary analytical method.

In summary, the unique manufacturing process herein provides a cost effective and direct method of enriching ebelin lactone from Bacopa monnieri, which may provide a more therapeutically effective plant-based ingredient for use in dietary supplements presented in a pharmaceutical form (e.g., tablet, capsule, powdered drink, etc).

### Example 2: Bioassay of Acid-Hydrolyzed Extract

Two different versions of the processed extract were analyzed by bioassay. The first extract was the result of a less complete hydrolyzation, which resulted in higher levels of jujobogenin and pseudojujobogenin. The hydrolyzation process occurs in two stages (FIG. 2).

When the hydrolyzation step is only done partially, significant amounts of jujubogenin and pseudojujubogenin exist in the extract. On the other hand, when the hydrolyzation process is more complete, then the extract will contain negligible amounts of jujubogenin and pseudojujubogenin, while containing much higher levels of ebelin lactone and bacogenin A1.

The bioassay results showed clear differences between the incomplete hydrolyzation extract and the complete hydrolyzation extract. The incomplete hydrolyzation extract yielded subjective effects that were closer in character to Bacopa, whilst displaying an improved pharmacokinetic profile when compared to non-hydrolyzed Bacopa monnieri. The effects appear much quicker and are less lethargy inducing when compared to non-hydrolyzed Bacopa. There was also a perceptible difference in the character of effects when compared to non-hydrolyzed Bacopa monnieri extracts, which is due to the already high concentration of ebelin lactone and bacogenin A1 in the partially hydrolyzed extract. However, the effects are being “muddied” by the remaining jujobogenin and pseudojujubogenin.

The fully hydrolyzed extract presented a very different set of subjective effects when compared to non-hydrolyzed and partially hydrolyzed Bacopa monnieri extracts. The classic Bacopa monnieri effects, particularly the calming and lethargy inducing effects, are now no longer present in the fully hydrolyzed Bacopa monnieri extract. Instead, the extract presents itself with a much more selective and subjectively “clean” effects profile. Rather than being lethargy inducing, the fully hydrolyzed extract is actually somewhat stimulating.

The bioassay results demonstrated the presence of different constituents of Bacopa monnieri and the types of subjective effects they produce in humans. Remarkably, the results herein show acute subjective effects of the present extract composition and illustrate the function of each constituent in the brain and body. Further, contents of the naturally occurring bacosides in the extract compositions can be manipulated by the present hydrolyzation process.

More significantly, the overall effect profiles of the bacoside compositions can be controlled by precisely controlling the degree of hydrolyzation in the present methods. In particular, partial (or less) hydrolyzation may result in more calming extracts that may serve as anxiolytic and sleep promoting agents. On the other hand, complete hydrolyzation may provide a more selective, cognitively enhancing agent.

While the partially hydrolyzed Bacopa monnieri extract may be identified as a Bacopa monnieri extract in a blind test by an expert, the fully hydrolyzed extract could not. That is, without the present method, the effects profile in the fully hydrolyzed extract would not exist. Thus, the present method created an entirely new subjective experience with fully hydrolyzed Bacopa monnieri extract.

### Example 3: Analysis of Ebelin Lactone

Analytical detection and quantitation of ebelin lactone. The Woodward-Fieser (WF) rules (Equation 1) were used to calculate the λₘₐₓ of the ebelin lactone product, as shown below.

λₘₐₓ=Base value+ΣSubstituent Contributions+ΣOther Contributions

### Equation 1: Summary Description of Woodward-Fieser Rules

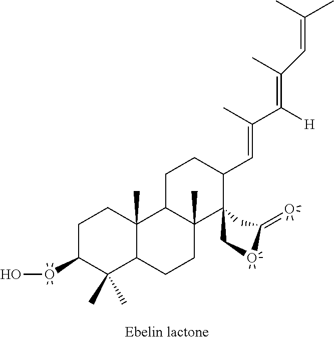 [0165] Base value: 214 nm (acyclic base)
Substituent contributions: +30 nm (double bond conjugation extension), +20 nm (four alkyl substituents at +5 nm each)
Other Contributions: Solvent effect unknown, Steric strain unknown
Theoretical λₘₐₓ: 264 nm
Empirical λₘₐₓ: 278 nm

A 14 nm discrepancy was observed between the theoretical and empirical λₘₐₓ. It is hypothesized that the molecule likely has some amount of steric strain, possibly due to the lactone structure itself.

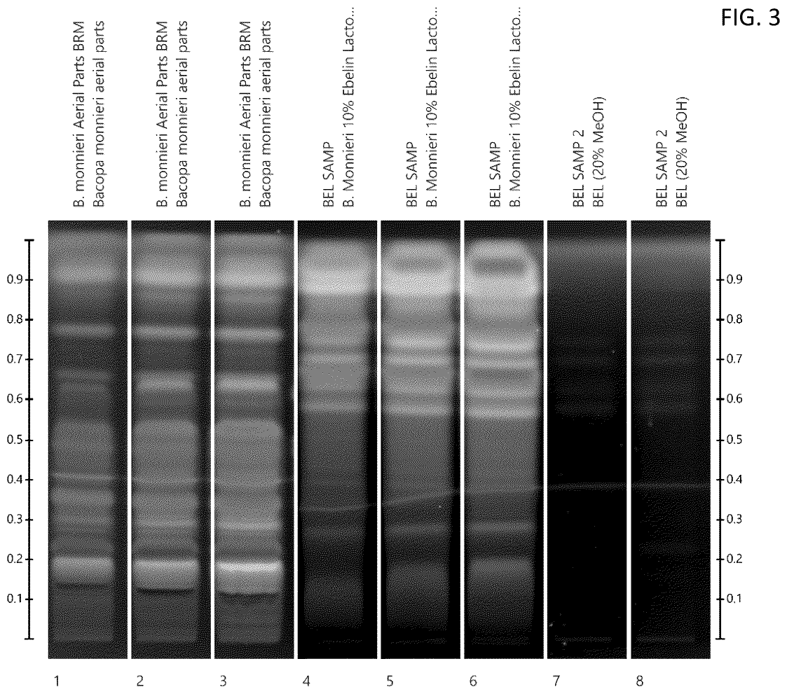

HPTLC identification of an ebelin lactone standardized extract. Ebelin lactone in the current product shows a strong differentiation from Bacopa monnieri aerial parts, when tested via High Performance Thin Layer Chromatography (HPTLC). As shown in a representative chromatographic plate in FIG. 3, the ebelin lactone product (lanes 4-8) shows a significant difference in bands in the lower regions of the plate as compared to the authenticated botanical reference material, Bacopa monnieri aerial parts (lanes 1-3). The upper region of the plate is also highly differentiated, with the ebelin lactone product showing intense peach and orange bands where the reference material shows few, faint green, tan, and fuchsia bands.

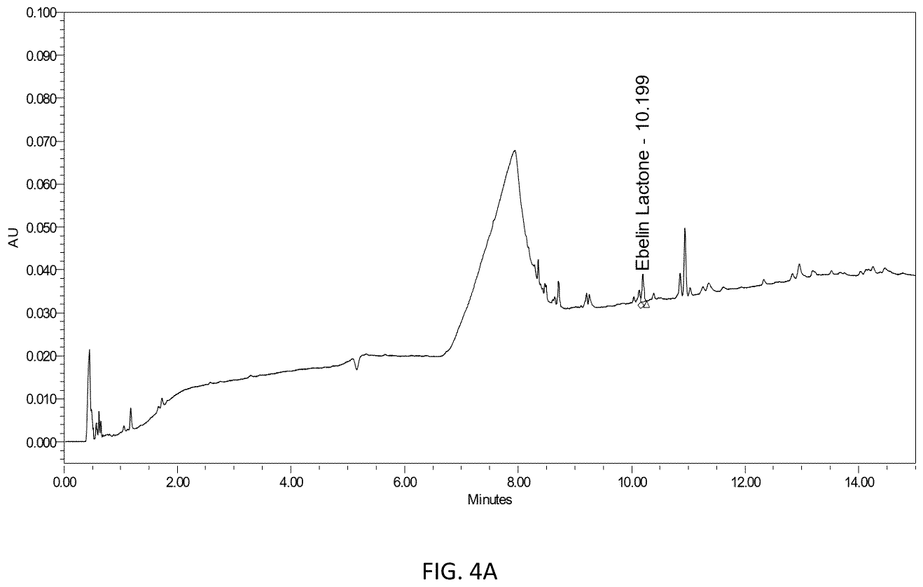

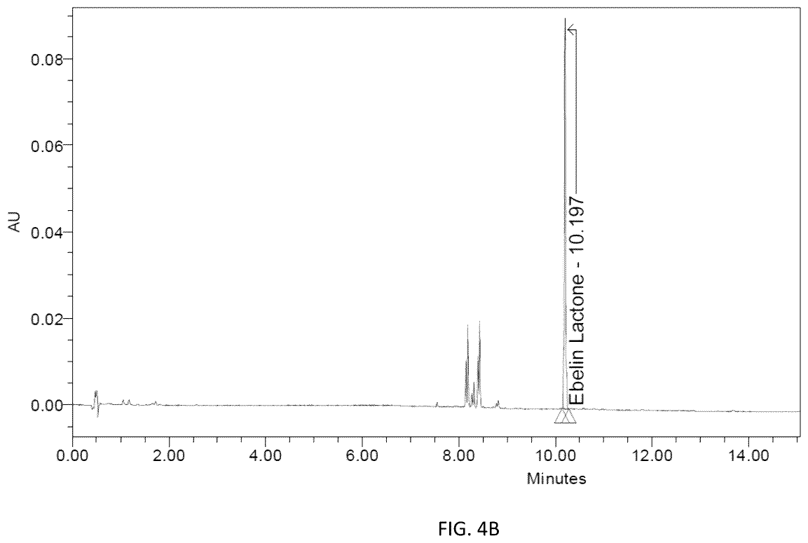

Analytical quantitation of ebelin lactone via UPLC. For quantitation of the ebelin lactone product, approximately 500 mg of the sample is dissolved in approximately 25 mL of methanol in a 50 mL volumetric flask. The sample is then vortexed, sonicated at 40° C. for 10 minutes, filtered with a nylon 0.22 μm filter, diluted to the mark, and then diluted 10-fold with calibrated pipettors. Using a primary standard of bacopaside A3 with 95%+purity, approximately 1 mg is added to a 10 mL volumetric flask and approximately 5 mL of methanol is added. The standard is then sonicated at room temperature for 10 minutes. The flask is the removed, diluted to the mark, and filtered with a nylon 0.22 μm into an autosampler vial. The sample and standard are then chromatographed on a reverse phase C18 column (Acquity BEH 100×2.1 mm, 1.7 μm) and the sample peak at ˜10.2 minutes quantitated against the bacopaside A3 standard at 205 nm. A one-point external calibration model or a multi-point external calibration model is used for the quantitation, and USP <731> loss on drying is used to determine the water content for the dry basis calculation, which is then used to determine the final assay value. The chromatograms at 205 nm and 278 nm are shown in FIG. 4A and FIG. 4B, respectively.

Each publication, patent, and patent publication cited in this disclosure is incorporated in reference herein in its entirety. The present invention is not intended to be limited to the foregoing examples, but encompasses all such modifications and variations as come within the scope of the appended claims.

1. A method for preparing an ebelin lactone or bacogenin A1 enriched composition, the method comprising contacting an acid-hydrolyzed extract of Bacopa monnieri with an antisolvent, thereby precipitating the ebelin lactone or bacogenin A1 enriched composition.

2. The method of claim 1, wherein contacting the acid-hydrolyzed extract with the antisolvent increases pH.

3. The method of claim 1, wherein the antisolvent is an aqueous antisolvent.

4. The method of claim 1, further comprising drying and milling the precipitate.

5. The method of claim 4, wherein the dried and milled precipitate has an ebelin lactone content of at least 10%.

6. The method of claim 1, wherein the acid-hydrolyzed extract of Bacopa monnieri is prepared by:
washing pulverized Bacopa monnieri thereby forming a wash-insoluble fraction;
drying the wash-insoluble fraction to a moisture content below a predetermined value;
extracting, with a polar protic extractant, bacosides from the dried wash-insoluble fraction;
concentrating the extract, wherein the concentrated extract has a bacoside content above a predetermined value;
hydrolyzing the concentrated extract with an acidified solvent, thereby forming the acid-hydrolyzed extract of Bacopa monnieri.

7. The method of claim 6, wherein contacting the acid-hydrolyzed extract with the antisolvent increases pH.

8. The method of claim 6, wherein the antisolvent is an aqueous antisolvent.

9. The method of claim 6, wherein the acidified solvent comprises a polar protic solvent and a mineral acid.

10. The method of claim 9, wherein the polar protic solvent is methanol or ethanol.

11. The method of claim 9, wherein the mineral acid is sulfuric acid.

12. The method of claim 6, wherein the predetermined moisture content is at least 15%.

13. The method of claim 6, wherein the polar protic extractant comprises methanol.

14. The method of claim 6, wherein the predetermined bacoside content is at least 20%.

15. The method of claim 6, wherein the extract is concentrated by drying.

16. The method of claim 6, wherein the pulverized Bacopa monnieri is washed with an aqueous wash.

17. The method of claim 6, wherein the extract is filtered prior to concentration.

18. The method of claim 1, wherein the acid-hydrolyzed extract of Bacopa monnieri is prepared by:
washing, with an aqueous wash, pulverized Bacopa monnieri, thereby forming a wash-insoluble fraction;
drying the wash-insoluble fraction to a moisture content below 15%;
extracting, with a polar protic extractant comprising methanol, bacosides from the dried wash-insoluble fraction;
filtering the extract,
concentrating the filtered extract, wherein the concentrated extract has a bacoside content above 20%;
hydrolyzing the concentrated extract with an acidified solvent comprising a polar protic solvent and a mineral acid, thereby forming the acid-hydrolyzed extract of Bacopa monnieri.

19. The composition prepared by the method according to claim 1.

20. The composition of claim 19, wherein the precipitate after drying has an ebelin lactone content of at least 10%.

21. The composition prepared by the method according to claim 6.

22. The composition of claim 21, wherein the precipitate after drying has an ebelin lactone content of at least 10%.

23. The composition prepared by the method according to claim 18.

24. The composition of claim 21, wherein precipitate after drying has an ebelin lactone content of at least 10%.

25. A pharmaceutical composition comprising an effective amount of the composition according to claim 19 and a pharmaceutically acceptable carrier.

26. A nutraceutical composition comprising the composition according to claim 19 and a nutraceutically acceptable carrier.

27. A method of treating a subject in need of ebelin lactone or bacogenin A1, the method comprising administering an effective amount of the composition according to claim 19 to the subject.
